(19) United States
(12) Patent Application Publication
(10) Pub. No.: US 2026/0051390 A1
(43) Pub. Date: Feb. 19, 2026

(54) Artificial Intelligence Management of Emergency Responders

(71) Applicant: Govermentjobs.com, Inc. DBA NEOGOV, El Segundo, CA (US)

(72) Inventors: Shane Nicholas Evangelist, Culver City, CA (US); Jonathan Michael Adler, Los Angeles, CA (US); Filip Novoselec, Zapresic (HR); Yogen Sunilbhai Darji, Vadodara (IN); Scott Michael MacDonald, Seward, NE (US); Stephen William Green, Poughkeepsie, NY (US); Alexander Henry Montgomery, San Diego, CA (US); Seng Fong Chang, Los Angeles, CA (US); Mario Jambrovic, Zagreb (HR)

(73) Assignee: Govermentjobs.com, Inc. DBA NEOGOV, El Segundo, CA (US)

(21) Appl. No.: 18/808,050

(22) Filed: Aug. 18, 2024

(51) Int. Cl. G16H 20/70; G16H 50/20

(52) U.S. Cl. CPC G16H 20/70; G16H 50/20

(57) ABSTRACT

The disclosed solution is generally configured for integration with a computer aided dispatch (“CAD”) system in order to analyze the mental and emotional health of law enforcement officers. The disclosed solution relies on artificial intelligence (“AI”) based on large-language models (“LLMs”) in order to process and categorize CAD-based data in order to detect opportunities to provide mental and emotional support for responding law enforcement who face difficult emergency situations. The disclosed solution is configured to operate with existing CAD systems in order to reduce reconfiguration and retraining of dispatchers.

### BACKGROUND

Emergency responders are responsible for maintaining public safety and order. As part of this responsibility, emergency responders are subjected to the most extreme types of stress of any profession in society. Law enforcement officers, in particular, enter situations where they must manage the safety of several individuals who are acting in very disparate manners. For instance, the responding officer is charged with protecting victims but also the actual subject (or “suspect”). Stated differently, an officer is balancing the needs of many people in extremely dangerous situations, thus leading to mental and emotional stress on the officer.

The problem of protecting people is further compounded by the role of the officer who must also protect other first responders-such as firefighters, medical responders, as well as other responding officers. A simple situation is illustrative. A subject steals a vehicle with a driver still inside the vehicle (i.e., a carjacking). When pursued by law enforcement officers, the subject strikes other pedestrians with the vehicle, causing severe bodily injury. Additionally, the subject strikes another vehicle that ignites into flames. Shortly thereafter, the subject loses control of the vehicle and hits the brick wall of a building. The subject then exits the vehicle and holds the driver hostage with a firearm. A long standoff between law enforcement and the subject ensues.

In the above-stated situation, medical workers will be required to respond to the injured pedestrians, and law enforcement will need to ensure their safety while the armed subject still poses a danger to the driver and other bystanders. Further, the responding officers will be responsible for protecting one another during the incident until backup arrives. Fire crews arrive on the scene and begin extinguishing the fires in nearby burning vehicles while evacuating drivers and passengers.

The salient point from the entire incident is law enforcement responders are subjected to the stress of witnessing the pain and death of innocent people. Further, law enforcement are human beings who make mistakes and can be responsible, inadvertently, for causing the deaths of innocent people. For example, law enforcement officers may mistake a victim for a subject during the use of lethal force. Simply stated, the stress responding officers incur is almost unimaginable and indescribable.

The stress of these situations incurs a cost on first responders. In many cases, responding officers resort to self-help that is often unhealthy. For example, tragically, responding officers may abuse alcohol after work to cope with the stress. This type of self-help causes further suffering in the responding officer that may lead to personal problems such as divorce, gambling, absenteeism, loss of personal connections, suicidal ideation, etc. Simply stated, self-help is suboptimal.

The complex situation described above is extreme in nature and easily identified by supervisors as causing stress. As such, responding officers may receive counseling by professionals in order to work through the stress of the carjacking situation above. But the question remains how to identify an officer who needs mental and emotional assistance for cumulative stress.

Cumulative stress is different in nature. An officer who responds to moderately severe emergencies day after day may not be noticed by supervisors as needing help. For example, responding to child abuse emergencies is not necessarily stressful when distributed over the course of six months. However, if an officer responds to five child abuse emergencies and ten graphic batteries over the course of two weeks, then the officer is likely to need counseling. The question then becomes how does a supervisor notice the smaller events as becoming cumulative to the stress placed on the officer. Unlike the carjacking above, the officer who works smaller emergencies consistently may be overlooked by supervisors until it is too late to intervene.

What is needed are systems and methods that leverage artificial intelligence (and large-language models) to detect cumulative stress placed on officers in order to improve the mental and emotional health (i.e., wellness) of first responders, including law enforcement officers. As such, cumulative stress can be detected earlier in order to provide early intervention to help the mental state of the officer.

### BRIEF DESCRIPTION OF DRAWINGS

The accompanying drawings, which are incorporated herein and constitute part of this specification, illustrate exemplary aspects of the claims, and together with the general description given above and the detailed description given below, serve to explain the features of the claims.

FIG. 1 depicts a block diagram of a responder wellness system.

FIG. 2A depicts a block diagram of computer aided dispatch (“CAD”) incident data.

FIG. 2B depicts a block diagram of large language model (“LLM”) prompt data.

FIG. 2C depicts a block diagram of awareness system data.

FIG. 3A depicts a block diagram of a CAD system.

FIG. 3C depicts a block diagram of an awareness system.

FIG. 4A depicts a flowchart of a process associated with a responder wellness system.

FIG. 4B depicts a flowchart of a process associated with a responder wellness system.

FIG. 4C depicts a flowchart of a process associated with a responder wellness system.

FIG. 5A depicts a flowchart of a process associated with a responder wellness system.

FIG. 5B depicts a flowchart of a process associated with a responder wellness system.

FIG. 6 depicts a block diagram of a user interface associated with a responder wellness system.

FIG. 7A is a block diagram illustrating an example computing device suitable for use with the various aspects described herein.

FIG. 7B is a block diagram illustrating an example server suitable for use with the various aspects described herein.

### DETAILED DESCRIPTION

Various aspects will be described in detail with reference to the accompanying drawings. Wherever possible, the same reference numbers will be used throughout the drawings to refer to the same or like parts. References made to particular examples and implementations are for illustrative purposes, and are not intended to limit the scope of the claims.

The disclosed solution is generally configured to provide early detection of stress incurred by law enforcement officers-whether that stress is a singular event or cumulative of several events. As stated above, the cumulative stress of law enforcement work leads to negative health outcomes for law enforcement officers. By handling and simply witnessing emergency events, law enforcement officers are subjected to some of the most extreme stressors of any profession. Stated differently, a “bad day at work” for a law enforcement officer is much different than the same of a banker, since the law enforcement officer is dealing with real-life emergencies that often include injuries and death. Moreover, several “bad days at work” can lead to negative outcomes for officers.

Mental health is often addressed by a combination of well-established psychological and wellness approaches. For example, psychologists may have therapeutic sessions with law enforcement officers after witnessing the death of a child in a vehicle collision. Another example is with veteran law enforcement officers who do not necessarily need psychological counseling but rather some time away from work—in which case some personal days away from work may be all that is required to maintain mental health.

Identifying when and how to improve the wellness of officers is non-trivial. While every individual is unique, there are well-established understandings in the field that certain events are more traumatic than others. For instance, an officer who pursues a fleeing shoplifter will not experience as much stress as an officer who is required to use lethal force to neutralize a subject. Further, stress is often cumulative. While one difficult event (e.g., a homicide) is traumatic, an officer who only has one over the course of 6 months will not be as affected as an officer who sees a homicide every week for the same period.

The disclosed solution generally provides a system and method for integrating software into existing computer aided dispatch (“CAD”) systems to gather and analyze (using artificial intelligence (“AI”)) emergency events in order to ascertain the cumulative stress incurred by the responding officers. CAD systems are generally those used by dispatchers to communicate with responding officers as well as memorialize the events of the emergency. For example, a dispatcher may alert an officer about an emergency related to vehicular theft. The response of the officer is recorded in the CAD system by the dispatcher. The dispatcher will continue to communicate with the officer while the officer reports the details of the theft (e.g., interviewing witnesses). These details are often recorded in CAD notes. Further, responding officers may augment CAD notes with field notes (e.g., using a smartphone-based application).

The disclosed solution relies on large language models (“LLMs”), a form of AI, to perform artificial intelligence-based analysis of tens of thousands of CAD-based events that are stored in CAD notes (and associated other types of data/fields). The LLM is configured in advance to find and analyze the types of stress that an officer experiences from different events. Simply understanding the type of event (e.g., burglary) is often insufficient to determine what types of events the officer witnessed. For instance, CAD notes may indicate that the burglary was a simple theft inside a detached garage which is technically a burglary; however, no sleeping people were inside. In contrast, a burglary of a home where people are sleeping and graphically assaulted as part of the burglary will be recorded in the CAD notes such that the LLM may capture those details to understand that the responding officer has witnessed a more stress-inducing burglary. As such, the same type of event (i.e., the burglaries) are very different when viewed as causing stress on the responding officer. Again, such differences are recorded in the CAD notes and analyzed by the disclosed solution.

Once the LLM has categorized the nature of the incidents, the disclosed solution uses this categorized data to present human supervisors with data and analytics sufficient to identify mental and emotional risks to officers who handled the incident. As stated, many well-established methods of addressing trauma exist (e.g., counseling, administrative leave, etc.). However, the identification of when the officer needs addressing of this stress is lacking in the field. Therefore, the disclosed solution performs statistical and mathematical analysis to present supervisors with necessary information to identify and provide appropriate assistance to these officers.

The disclosed solution is configured to interoperate with existing CAD systems such that law enforcement agencies need not wholly replace existing systems. As such, dispatchers may simply perform their duties as always because the disclosed solution is configured to have the necessary data integrations with existing systems. Therefore, minimal (or likely no) additional training of dispatchers and responding officers is necessary. Further, the disclosed solution is configured to be readily utilized by supervisors to provide the well-established support for officers experiencing extreme emergency situations.

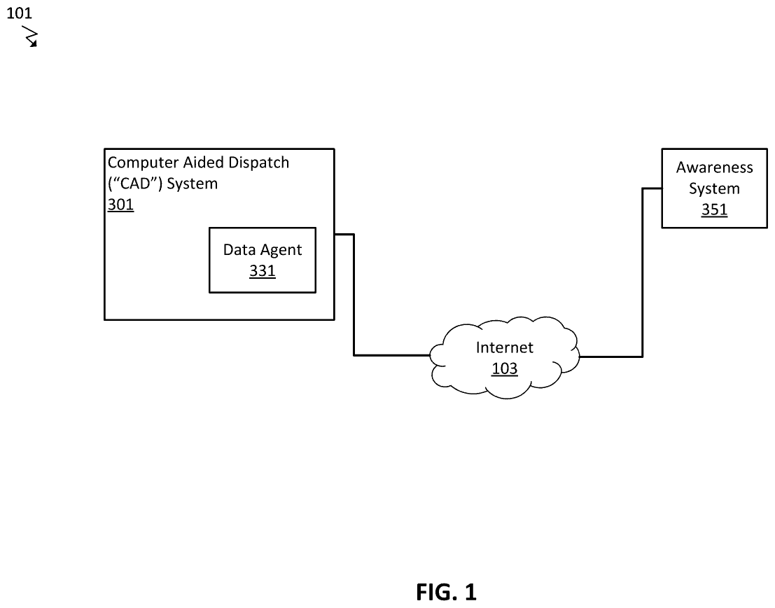

FIG. 1 depicts a block diagram of a responder wellness system 101. The responder wellness system 101 comprises a data agent 331 and an awareness system 351, both of which are connected to the Internet 103. The data agent 331 is connected to a computer aided dispatch (“CAD”) system 301.

The CAD system 301 is generally configured to aid dispatchers with the communication and memorialization of incidents encountered by responding law enforcement officers. The CAD system 301 is typically a legacy system that has been used by agencies for many years, by dispatchers who have specialized training on the CAD system 301. Responding officers may have remote access to the CAD system 301 via applications in smartphones, vehicles, etc. As such, the CAD system 301 may receive field notes from responding officers as well.

The data agent 331 is generally configured to integrate into the CAD system 301. As such, the data agent 331 is installed at the CAD system 301 in order to communicate the data received, at the CAD system 301, to the awareness system 351 (via the Internet 103). For example, the data agent 331 may gather CAD-based notes and transmit the CAD notes to the awareness system 351 for processing (e.g., analysis using AI and LLMs).

The awareness system 351 is generally configured to process, analyze, and present data gathered by the CAD system 301, as communicated by the data agent 331. In one aspect, the awareness system 351 performs, via an LLM, analysis that excludes sensitive data from long-term storage. For example, the identities of victims and subjects may be filtered out by an LLM to protect privacy, reduce legal risk, etc.

The awareness system 351 is further configured to provide a user interface to supervising officers in order to manage the wellness of responding officers. As stated, well-established approaches exist to help officers deal with stress. The awareness system 351 is a specially configured system that leverages both data integration and AI (and LLMs) to readily identify wellness risks to officers. As such, supervising officers may provide the necessary support for those affected responders.

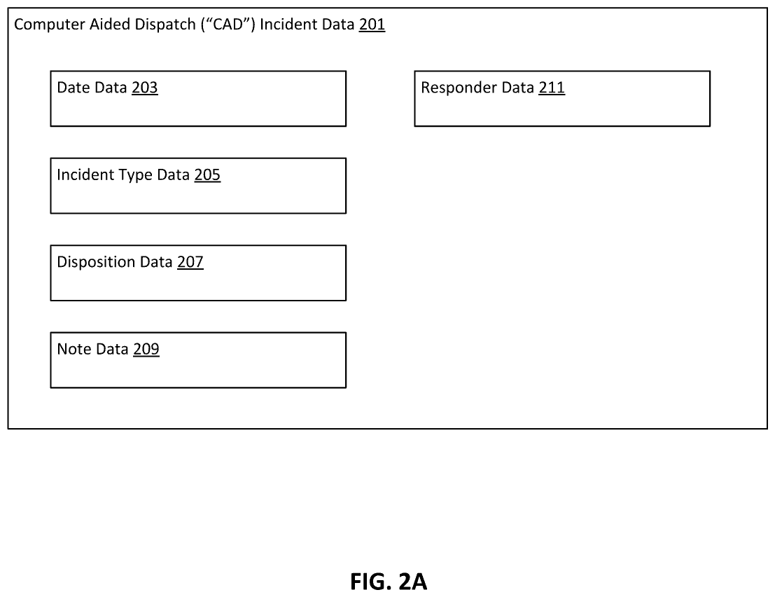

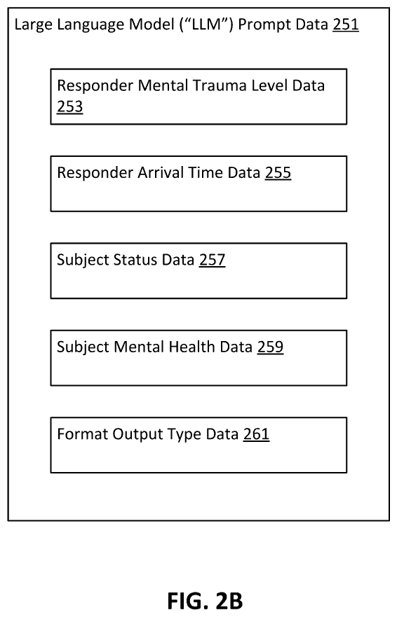

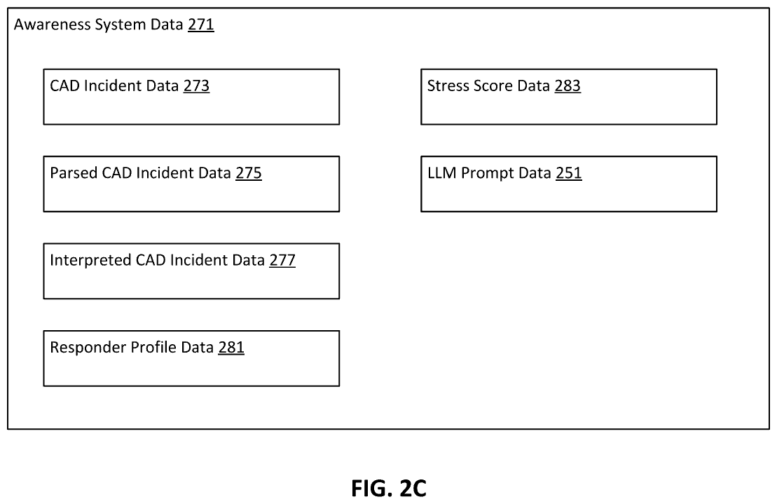

FIG. 2A depicts a block diagram of computer aided dispatch (“CAD”) incident data 201. The CAD incident data 201 is generally configured as a data structure to store relevant data associated with an incident (e.g., an emergency response to armed robbery). The CAD incident data 201 comprises date data 203, incident type data 205, disposition data 207, note data 209, and responder data 211.

The date data 203 is generally configured to store the date and time of the event. The date data 203 enables the awareness system 351 to capture the relationship between other events affecting the officer. For example, three days in a row of handling homicides is generally more traumatic than handling three homicides over the course of a year. As such, the LLM may account for proximity of time, via date data 203, when analyzing note data 209.

The incident type data 205 is generally configured to store the type of incident the officer has encountered. Some incident types include, but are not limited to: vehicular theft, larceny, battery, burglary, homicide, domestic violence, armed robbery, arson, disturbing the peace, public drunkenness, driving while intoxicated, etc. The type of incident is relevant to the awareness system 351 (and specifically the AI LLM) because different incidents generally have different levels of impact on officers. For example, handling larceny (e.g., shoplifting) is less stressful than handling a homicide with a child victim.

The disposition data 207 is generally configured to store the disposition of the incident. The details of the disposition data 207 are numerous and largely germane to the field of law enforcement and agency. Further, different agencies have different disposition types and codes. Nevertheless, the disclosed solution is configured to use LLMs to analyze any type of existing disposition in order to identify wellness risks to responding officers. Examples of dispositions are: arrest made (boarded and secured), cancelled, duplicate, no further action, stolen property (vehicle), unfounded, alarm call (cancelled by unit on scene), warning issued, citation issued, field interview conducted, no patrol available, assistance rendered, abandoned vehicle towed, K-9 search, code enforcement, report taken, referred to other agency, motorist assist, etc.

The note data 209 is generally configured to store the details of the incident (emergency) as recorded by the dispatcher operating the CAD system 301. Additionally, responding officers may add additional note data 209 to the CAD system 301 (e.g., via mobile devices). The dispatcher is responsible for notifying officers of the nature and location of an emergency, initially. During the response to the emergency, the responding officers and dispatcher are in constant communication in order to effectively respond to the incident. As part of this communication, the dispatcher records notes into the CAD system 301 which are stored as note data 209. Likewise, responding officers may add more details to the note data 209 based on events seen in the field.

The note data 209 may be free-text or structured-text with fields and a particular format. The note data 209 may be stored as HTML, XML, JSON, plain text, etc. The note data 209 may be human-readable, in one aspect.

The responder data 211 is generally configured to store information sufficient to identify the responding officer. For example, the responder data 211 may include: name, badge number, car number, rank, agency, height, weight, gender, service years, specialized training (e.g., special weapons, K-9, etc.), etc.

FIG. 2B depicts a block diagram of large language model (“LLM”) prompt data 251 that is used for prompt engineering. At a high level, prompt engineering is a set of carefully engineered and curated textual prompts that are given to an LLM as training material and/or instructions in order to cause the LLM to execute a particular task. As such, prompt engineering may or may not rely on structured data. One advantage of prompt engineering is the capability to configure an LLM using human-readable, human-understandable language.

The LLM prompt data 251 is generally configured to configure an LLM with parameters sufficient to process CAD incident data 201. As shown in the instant figure, the LLM prompt data 251 comprises several data types. However, these data types (e.g., responder trauma level data 253) are more representative of the kind of data that would be used as part of the prompt engineering associated with an LLM. In actual implementation, the data types of the LLM prompt data 251 may be more nebulous, overlapping and almost organic in nature because an LLM is generally configured using such type of communication rather than discrete data types, formulas, data structures, etc.

As disclosed at FIG. 2A, the note data 209 may be, in one aspect, free-text that is simply the real-time notes of a dispatcher who is in communication with a responding officer. As such, the note data 209 may vary widely between one dispatcher to the next. Further, officers may augment note data 209 with field notes. To address such variance in the CAD incident data 201, the LLM is required to have a configuration that provides for robust and effective analysis of the CAD incident data 201 without causing dispatchers (or responders) to adhere to a strict format.

As such, the LLM prompt data 251 comprises responder mental trauma level data 253, responder arrival time data 255, subject status data 257, subject mental health data 259, and format output type data 261. The responder mental trauma level data 253 is generally configured to classify the CAD incident data 201 into one of several categories that are generally associated with the real-world mental and emotional responses of officers to traumatic events. Table 1 below illustrates an example hierarchy of responder mental trauma level data 253.

TABLE 1
Responder Mental Trauma Levels

| Responder | |
|---|---|
| Mental Trauma | |
| Level | Events |
| High | Child fatality, juvenile family violence, infant fatality, |
| | response to mass shooting, mass casualty management, |
| | murder, suicide (graphic) |
| Medium | Graphic scene management, suicide investigation, |
| | response to shooting, officer-involved shooting, |
| | child victim |
| Low | Response to child abuse, motor vehicle accidents with |
| | fatalities, accidental deaths, trauma cleanup, force used |
| | against responder, aggression used against responder |
| None | Larceny, vandalism, minor in possession of alcohol, |
| | noise complaint |

The responder mental trauma level data 253 generally relates to the prompt engineering associated with the severity of mental stress associated with an incident. The LLM will weigh and classify the CAD incident data 201 into the various defined categories (as shown in Table 1 above). However, additional data may be applied to the responder mental trauma level data 253 to adjust for other real-world parameters, as will be stated below.

The responder arrival time data 255 is associated with responder mental trauma level data 253 and is configured to store the time and duration of the responder at the incident. For example, a shorter duration is likely to affect the officer less than an officer who has been present at a graphic scene for hours. In general, an emergency may be under more control than at the onset. As such, the responder arrival time data 255 is configured to configure the LLM to properly weigh and categorize the arrival time of the officer.

The subject status data 257 is associated with the responder mental trauma level data 253 and is generally configured to represent the status of the subject (e.g., suspect) for configuration of the LLM. For example, the LLM may be configured to reduce the weight (severity) of an incident wherein the subject has been arrested and secured in a holding facility.

The subject mental health data 259 is associated with the responder mental trauma level data 253 and generally configured to store the status of the mental state of the subject in order to configure the LLM. For example, the LLM may be configured to reduce the weight (severity) of an incident wherein the subject has a mental health issue that requires hospitalization. The general understanding by those in the field is that mental health patients act out of illness rather than malice.

The format output type data 261 is generally configured to enable the LLM to output any data related to the rationale of classification and weight of the responder mental trauma level data 253. For example, the format output type data 261 may include the responder mental trauma level data 253 (e.g., “high”) with a rationale as to why the LLM classified the CAD incident data 201 as such. An example rationale is shown as Rationale 1 below.

“Although the situation involved high stress factors such as the ex-girlfriend attempting to set the house on fire and being armed with a significant piece of iron, her being in possession of an aggressive object did not result in an immediate threat to the responders themselves, as she did not engage directly with them using the object. Additionally, by the time the subject was taken into custody, the immediate hostile circumstances had been mitigated. Therefore, the trauma impact on the responders is classified as ‘Low’ since the environment contained potential harm but did not escalate into direct violence against the responders. The early tension of potential violence and setting a fire, which can be traumatic, was counterbalanced by the successful de-escalation and containment of the situation, including extinguishing the fire without any ongoing danger by the time responders were managing the scene.”

### Rationale 1: Low Responder Mental Trauma Level Example

FIG. 2C depicts a block diagram of awareness system data 271. The awareness system data 271 is generally configured to store data associated with the operation and processing of the awareness system 351. The awareness system data 271 is, in part, data that is received from the data agent 331 associated with the CAD system 301. The awareness system data 271 comprises CAD incident data 273, parsed CAD incident data 275, interpreted CAD incident data 277, CAD analytics data 279, responder profile data 281, stress score data 283, and LLM prompt data 251.

The CAD incident data 273 is disclosed in more detail at FIG. 2A. In general, the CAD incident data 273 is generated at the data agent 331 and sent to the awareness system 351. However, to be clear, the CAD incident data 273 is further processed, updated, and stored by the awareness system 351, particularly as will be shown with respect to the processes disclosed herein.

The parsed CAD incident data 275 is generally configured to maintain a parsed instance of the CAD incident data 273. The parsed CAD incident data 275 is generally a more enriched version of the CAD incident data 273 such that users of the awareness system 351 have a more comprehensive view of the incident represented by the CAD incident data 273. In one aspect, the parsed CAD incident data 275 may be generated from the interpreted CAD incident data 277.

The interpreted CAD incident data 277 is generally configured to store data that has been processed by an LLM. The CAD incident data 273 is used as input to the LLM, and the interpreted CAD incident data 277 is the output. The interpreted CAD incident data 277 contains instanced information that corresponds to the responder mental trauma level data 253 within the LLM prompt data 251.

For example, assume the parsed CAD incident data 275 contains data related to a homicide as well as a burglary. The interpreted CAD incident data 277 will then contain data that has classified the parsed CAD incident data 275 as a “high” trauma event as embodied by the responder mental trauma level data 253.

At a high level, the CAD incident data 273 contains sensitive information related to subjects (e.g., name, birthdate, etc.). In contrast, the interpreted CAD incident data 277 is data that excludes sensitive information associated in the CAD incident data 273 (generally as received from the data agent 331). The removal of sensitive data is performed via an LLM that has been configured via prompt engineering (e.g., using the LLM prompt data 251).

The responder profile data 281 is generally configured to store various profiles of responders. For example, the responder profile data 281 contains data associated with real-world aspects of a responding officer (e.g., name, badge number, rank, etc.). In one aspect, the responder profile data 281 also contains previous mental health (wellness) data of the responder (e.g., previous counseling sessions).

The stress score data 283 is generally configured to represent a value of the mental and emotional status of a responding officer. Supervisors only have so many hours to perform law enforcement duties, which include the management of responding officers. As such, the stress score data 283 provides an elegant value that reflects the mental and emotion state (wellness) of any given responder. In one aspect, the stress score data 283 may be in a range of zero to one-hundred. However, one of skill in the art will appreciate that the stress score data 283 may be any range of scalar values.

The LLM prompt data 251 has been previously disclosed in more detail at FIG. 2B. The LLM prompt data 251, as shown in the instant figure, reflects an instance of the LLM prompt data 251 that would be used to configure an LLM. Therefore, one of skill in the art will appreciate that the LLM prompt data 251 may be tailored to the particular agency that is served by the awareness system 351.

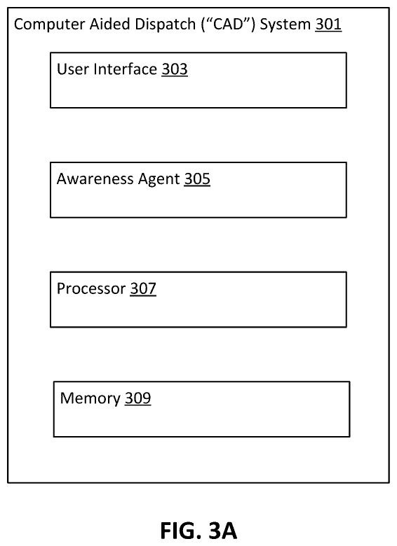

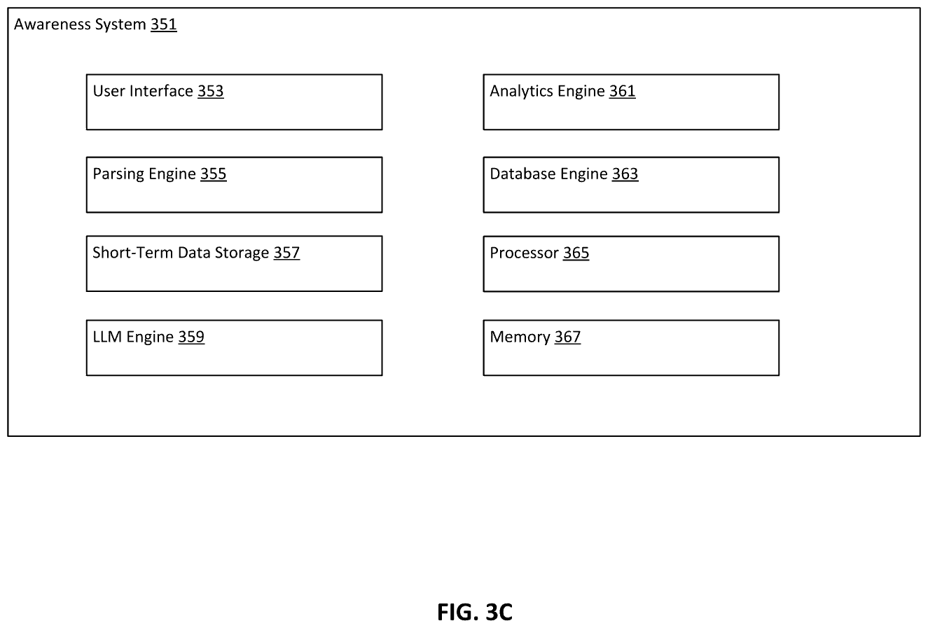

FIG. 3A depicts a block diagram of a CAD system 301. The CAD system 301 is generally a legacy system that is used by agencies to dispatch responders to a given emergency/incident. The CAD system 301, as shown, is configured for use with the awareness system 351, specifically via inclusion of the data agent 331. The CAD system 301 comprises a user interface 303, the data agent 331, a processor 307, and a memory 309.

The user interface 303 is generally configured to receive information as gathered by dispatchers. As stated, the user interface 303 is typically a legacy system that is utilized by dispatchers to enter information into the CAD system 301. The user interface 303 may be voice systems, keyboard, mouse, display, or a combination thereof. In one aspect, the user interface 303 may be a remote interface used by responding officers to enter field notes (e.g., via smartphone).

The data agent 331 is generally configured to communicate CAD incident data 201 to the awareness system 351. The data agent 331 is configured for installation at the CAD system 301 in order to maintain the legacy capabilities of the CAD system 301 as much as reasonably possible. The advantage of using the legacy capabilities is to reduce the resource cost to agencies deploying the responder wellness system 101. For example, dispatchers will require minimal (if no) additional training to use the responder wellness system 101.

The processor 307 may be a shared processor which is utilized by other systems, modules, etc. within the disclosed solution. For example, the processor 307 may be configured as a general-purpose processor (e.g., x86, ARM, etc.) that is configured to manage operations from many disparate systems, including the awareness system 351. The memory 309 is generally operable to store and retrieve information. The memory 309 may be comprised of volatile memory, non-volatile memory, or a combination thereof. The memory 309 may be closely coupled to the processor 307, in one aspect. For example, the memory 309 may be a cache that is co-located with the processor 307.

FIG. 3C depicts a block diagram of an awareness system 351. The awareness system 351 is generally configured to provide wellness-related information to supervisors based on the CAD incident data 201 as processed by the awareness system 351 (and associated processes disclosed herein). The awareness system 351 comprises a user interface 353, a parsing engine 355, short-term data storage 357, an LLM engine 359, an analytics engine 361, a database engine 363, a processor 365, and a memory 367.

The user interface 353 is generally configured to present information to supervisors. The user interface 353 may be a combination of voice systems, keyboard, mouse, and display. Such information may be embodied as the responder profile data 281 that is associated with the stress score data 283. The presentation of the responder profile data 281 includes the capabilities of interaction with the presented stress score data 283 such that supervisors may develop plans to address the data-driven mental and emotional state of responding officers.

The parsing engine 355 is generally configured to process the CAD incident data 201 into the parsed CAD incident data 275. In one aspect, the parsing engine 355 performs processing of the CAD incident data 201 in order to prepare the CAD incident data 201 for presentation to supervisors via the user interface 353.

The short-term data storage 357 is generally configured to store the CAD incident data 201 in a manner that reduces the risk of maintaining personally identifying information of subjects. One commercial example of short-term data storage 357 is an Amazon Web Services S3 Bucket. As stated above, the personal details of any given subject may be found in the CAD incident data 201. As such, the awareness system 351 maintains the CAD incident data 201 in the short-term data storage 357. The short-term data storage 357 provides a reduced-risk mechanism to avoid unduly maintaining/storing sensitive information. Therefore, the awareness system 351 has the capabilities to generate necessary wellness management of responding officers while maintaining personally identifying information for the minimal time necessary to achieve said result.

The LLM engine 359 is generally configured to operate on CAD incident data 201 in order to generate analytics related to the CAD incident data 201 (including generating stress score data 283). Specifically, the LLM engine 359 processes and interprets note data 209 to determine responder mental trauma level data 253. The LLM engine 359 is configured via the LLM prompt data 287. As such, the LLM engine 359 has the information necessary to analyze the CAD incident data 201, in general, with a focus on the note data 273. In one aspect, the LLM engine 359 may be OpenAI.

The analytics engine 361 is generally configured to generate analytics from the CAD incident data 201 (e.g., by analyzing interpreted CAD incident data 277). In one aspect, the analytics engine 361 is configured to generate stress score data 283 that is associated with responder profile data 281.

The database engine 363 is generally configured to store the interpreted CAD incident data 277. In comparison with the short-term data storage 357, the database engine 363 is intended to maintain longer-term storage of CAD-related data because the information is less sensitive than that contained within the note data 209. For example, the database engine 363 is configured to avoid storage of personally identifying and/or sensitive information in the note data 209 (e.g., name, social security number, etc.). In one aspect, the database engine 363 may be a MySQL database, an SQLite database, a PostgreSQL, etc.

The processor 365 may be a shared processor which is utilized by other systems, modules, etc. within the disclosed solution. For example, the processor 365 may be configured as a general-purpose processor (e.g., x86, ARM, etc.) that is configured to manage operations from many disparate systems. The memory 367 is generally operable to store and retrieve information. The memory 367 may be comprised of volatile memory, non-volatile memory, or a combination thereof. The memory 367 may be closely coupled to the processor 365, in one aspect. For example, the memory 367 may be a cache that is co-located with the processor 365.

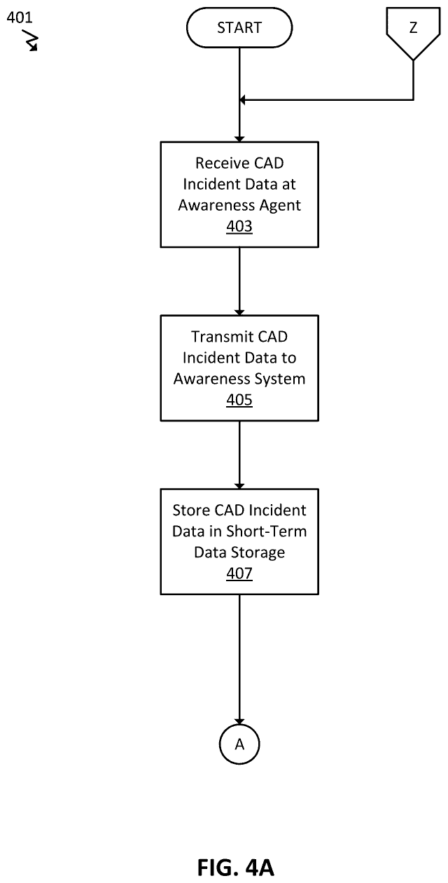

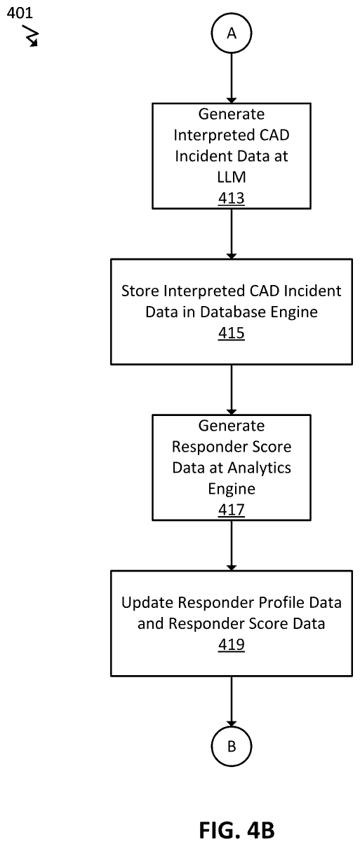

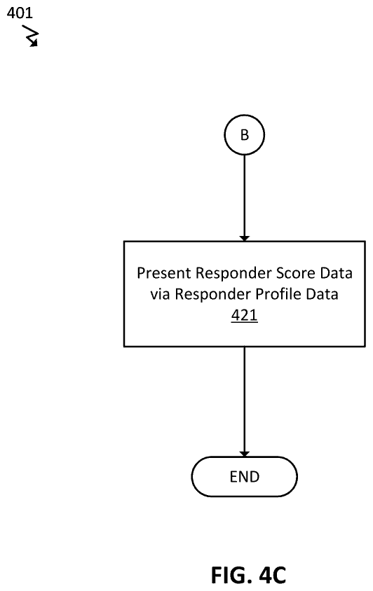

FIG. 4A depicts a flowchart of a process 401 associated with the responder wellness system 101. The process 401 is generally configured to receive information from the CAD system 301 in order to generate scoring of responding officers. Such scoring is configured to enable human supervisors to understand and execute well-established methods of reducing stress causing an undesirable score (e.g., stress score data 283).

The process 401 begins at the START block and proceeds to the step 403 where the process 401 receives CAD incident data 201 at the data agent 331. The CAD incident data 201 is generally entered by dispatchers at the CAD system 301. Typically, dispatchers are in communication with responding officers and record the events of the response in CAD notes (e.g., note data 209). Further, officers may add note data 209 via remote terminals/devices. The process 401 then proceeds to the step 405.

At the step 405, the process 401 transmits CAD incident data 201 to the awareness system 351. Once CAD incident data 201 is entered into the CAD system 301, the process 401 utilizes the data agent 331 to transmit the CAD incident data 201 to the awareness system 351. The process 401 then proceeds to the step 407.

At the step 407, the process 401 initially stores the CAD incident data 201 in the short-term data storage 373. Given the sensitive nature of CAD incident data 201, the process 401 stores such data in the short-term storage 373 in order to reduce the risk of exposing private information. Later, the awareness system 351 is configured to generate interpreted CAD incident data 277 and/or parsed CAD incident data 275 that is more focused on the wellness of the officer rather than the details of a particular incident. Stated differently, the identity of third parties involved in an incident are less relevant to the mental and emotional health of an officer. The process 401 proceeds to the Reference A.

FIG. 4B depicts a flowchart of the process 401 associated with the responder wellness system 101. The process 401 proceeds from the Reference A to the step 413. At the step 413, the process 401 generates interpreted CAD incident data 277 at the LLM engine 359.

The interpreted CAD incident data 277 is such that personally identifying information has been filtered and/or abstracted away. It is important to note that the process 401 is initially operating from the short-term data storage 373 (which stores the CAD incident data 201), since the awareness system 351 is configured to generate useful analytics with little (if no) personally identifying information. For example, the name and address of a subject may be processed and filtered out in resulting interpreted CAD incident data 277.

In one aspect, the interpreted CAD incident data 277 is processed to remove superfluous information. For example, the dispatcher may include information that is either superfluous such as the make and model of a vehicle and the name of the owner. As such, those types of details are not particularly useful to render actionable analytics (e.g., the stress score data 283). As such, the process 401 may remove such types of details when generating the interpreted CAD incident data 277. The process 401 then proceeds to the step 415.

At the step 415, the process 401 stores the interpreted CAD incident data 277 in the database engine 363. The interpreted CAD incident data 277 is processed such that identifying personal information has been removed (or abstracted) such that there is minimized risks of retaining incident-related information in the long term. Stated differently, the interpreted CAD incident data 277 is processed such that the wellness of the officer may be clearly determined based on further analytics processing. The process 401 then proceeds to the step 417.

At the step 417, the process 401 processes the interpreted CAD incident data 277 to generate stress score data 283. In one aspect, the stress score data 283 may be derived from parsed CAD incident data 275 that is a further enriched/processed instance of interpreted CAD incident data 277 via the parsing engine 355. In another aspect, the interpreted CAD incident data 277 is such that the nature and severity of the incident is organized into a useful data structure already. At the instant step, the process 401 associates the particular event with the personalized scoring of the responding officer. For example, if an officer has already experienced several recent “high” level events (e.g., homicide), then the analytics engine 361 is configured to account for recent incidents in order to generate a properly weighted score for the officer (as stress score data 283). The process 401 then proceeds to the step 419.

At the step 419, the process 401 updates the responder profile data 281 and the responder profile score data 283. Each responding officer has a responder profile data 281 instance which contains various personalized details about the officer (e.g., name, badge number, rank, etc.). Further, the responder profile data 281 may comprise previous incidents that the officer has encountered-including any relevant responder profile score data 283. Changes to the responder profile data 281 and the responder profile score data 283 are accounted for at the instant step. In one aspect, these updates occur at the database engine 363. The process 401 then proceeds to the Reference B which is continued at FIG. 4C.

FIG. 4C depicts a flowchart of the process 401 associated with the responder wellness system 101. The process 401 continues at the Reference B and proceeds to the step 421. At the step 421, the process 401 presents stress score data 283 via responder profile data 281. As stated, the supervisor may review the stress score data 283 at the user interface 353 of the awareness system 351. Based on the information presented, the supervisor can take appropriate action to improve the wellness of the officer (e.g., by recommending counseling, offering administrative leave, etc.). The process 401 then proceeds to the END block and terminates.

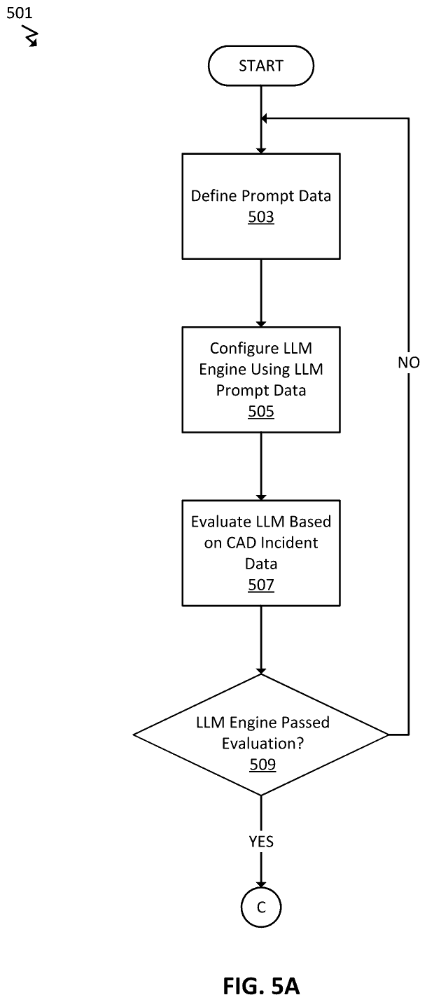

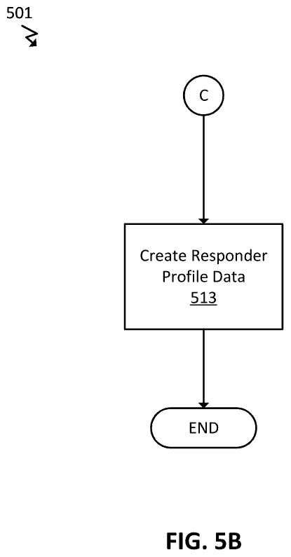

FIG. 5A depicts a flowchart of a process 501 associated with the responder wellness system. The process 501 is generally configured to process CAD incident data 201 in order to provide CAD-related analytics via a configuration of the responder wellness system 101. The process 501 begins at the START block and proceeds to the step 503.

At the step 503, the process 501 defines LLM prompt data 251. The LLM prompt data 251 is described in detail at FIG. 2B. In the instant step, an instance of the LLM prompt data 251 would be designed by system operators of the responder wellness system 101 in order to properly categorize the CAD incident data 201 as relating to a particular category (e.g., as those found in Table 1 above). The process 501 proceeds to the step 505.

At the step 505, the process 501 configures the LLM engine 359 using LLM prompt data 287. The LLM prompt data 287 is presented as input to the LLM engine 359. The LLM engine 359 then configures the output of the large-language model to detect and analyze CAD incident data 201. The process 501 then proceeds to the step 507.

At the step 507, the process 501 evaluates the output of the LLM engine 359 based on the CAD incident data 201. To be clear, the instance of CAD incident data 201 referenced at this step may be test data used to validate the inputs and outputs of the LLM engine 359. In one aspect, a rationale of analysis may be stored in the format output type data 261 and may be reviewed in order to determine that the LLM engine 359 is indeed processing and analyzing the CAD incident data 201 properly. The process 501 then proceeds to the decision block 509.

At the decision block 509, the process 501 determines whether the LLM engine 359 passes the evaluation of CAD incident data 201. In one aspect, an evaluation may be considered passed when the categorizations adhere to well-established norms for addressing the wellness of responding officers. For example, a homicide should be typically categorized as a “high” trauma event instead of a “low” trauma event. If the evaluation is not passed, the process 501 proceeds along the NO branch to the step 503. If the evaluation passes, the process 501 proceeds along the YES branch to the Reference C.

FIG. 5B depicts a flowchart of the process 501 associated with the responder wellness system 101. The process 501 continues at the Reference C and proceeds to the step 513. At the step 513, the process 501 creates responder profile data 281. When the awareness system 351 is configured, there may need to be accounts and/or profiles established for responders who are monitored by the awareness system 351 using AI. In one aspect, the instant step may be utilized to update existing responder profile data 281 as well. The process 501 then proceeds to the END block and terminates.

Turning back to FIG. 4A, the Reference Z indicates that the process 501 may precede the invocation of the process 401. For instance, the process 501 may be executed in order to properly configure the awareness system 351, specifically the LLM engine 359. Thereafter, the process 401 may properly execute the required functionality to analyze CAD incidents that result in risks to the mental health of first responders. One of skill in the art will appreciate that the process 501 may be repeated as necessary to adapt to situations at a given law enforcement agency. For example, the categorizations of responder mental trauma level data 253 may be adjusted if a particular agency has many new recruits who may be more sensitive to difficult emergencies/incidents.

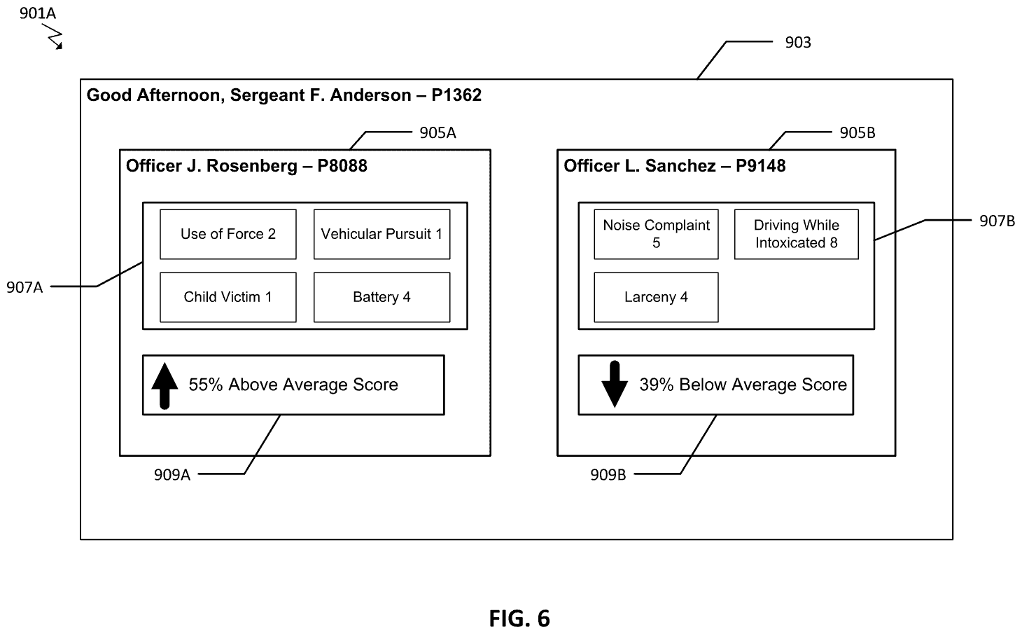

FIG. 6 depicts a block diagram of a user interface 901A associated with a responder wellness system. The user interface 901A is presented by the awareness system 351. As shown, the greeting at the top is directed to a supervisor, namely Sergeant F. Anderson. The supervisor can view the profiles of various law enforcement personnel (e.g., responder profile data 281) and readily determine high-level details about responder wellness (e.g., as stress score data 283).

The user interface 901A comprises a first profile 905A and a second profile 905B. The first profile 905A comprises a plurality of tags 907A and a score indicator 909A. The second profile 905B is similarly configured. The profile 905A shows the name of the responder Officer J. Rosenberg. The presented data corresponds to that stored in the responder profile data 281. The plurality of tags 907A present a high-level view of salient CAD incidents that the officer has encountered during a given period of time. The plurality of tags 907A may represent the data stored in the interpreted CAD incident data 277 as processed by the analytics engine 361.

The score indicator 909A is higher than that of the score indicator 909B because the plurality of tags 907A has higher-level traumatic events than the plurality of tags 907B. In other words, the events in Table 1 above are shown as a simple scalar value in the score indicators 909A, 909B. Further, trends of scoring are shown in the score indicators 909A, 909B to account for the responder profile data 281 as being processed by the analytics engine 361.

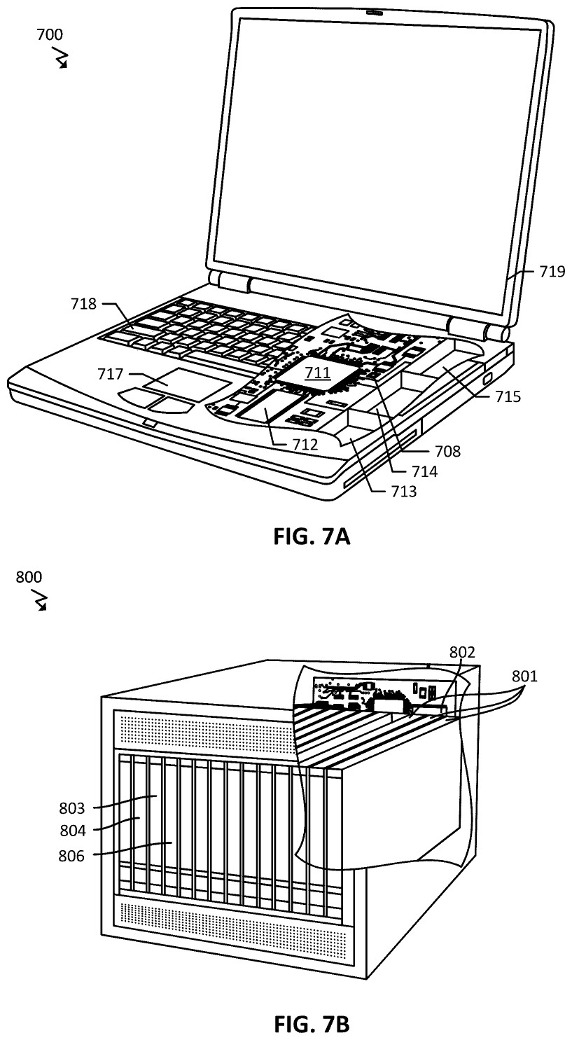

FIG. 7A is a block diagram illustrating a computing device 700 suitable for use with the various aspects described herein. The computing device 700 may include a processor 711 (e.g., an ARM processor, an x86 processor, etc.) coupled to volatile memory 712 (e.g., DRAM) and a nonvolatile memory 713 (e.g., a flash memory). Additionally, the computing device 700 may have a network access interface 708 for communication with network (e.g., the Internet, a local area network, etc.). The computing device 700 may also include an optical disk drive 714 and/or a removable disk drive 715 (e.g., removable flash memory) coupled to the processor 711. The computing device 700 may include a touch surface 717 that serves as a user interface for the computing device 700, whereby the computing device 700 may receive drag, scroll, flick etc. gestures similar to those implemented on computing devices equipped with a touch screen display. In one aspect, the touch surface 717 may be integrated into one of the components of the computing device 700 (e.g., a display 719). In one aspect, the computing device 700 may include a keyboard 718 which is configured to accept user input.

FIG. 7B is a block diagram illustrating a server 800 suitable for use with the various aspects described herein. In one aspect, the server 800 may be part of a cloud computing network. The server 800 may include one or more processor assemblies 801 (e.g., an x86 processor) coupled to volatile memory 802 (e.g., DRAM) and a nonvolatile memory 804 (e.g., a magnetic disk drive, a flash disk drive, etc.). As illustrated in instant figure, processor assemblies 801 may be added to the server 800 by insertion into the racks of the assembly. The server 800 may also include an optical disk drive 806 coupled to the processor 801. The server 800 may also include a network access interface 803 (e.g., an ethernet card, WIFI card, etc.) coupled to the processor assemblies 801 for establishing network interface connections with a network (e.g., the Internet, a 5G network, etc.).

The foregoing method descriptions and diagrams/figures are provided merely as illustrative examples and are not intended to require or imply that the operations of various aspects must be performed in the order presented. As will be appreciated by one of skill in the art, the order of operations in the aspects described herein may be performed in any order. Words such as “thereafter,” “then,” “next,” etc. are not intended to limit the order of the operations; such words are used to guide the reader through the description of the methods and systems described herein. Further, any reference to claim elements in the singular, for example, using the articles “a,” “an,” or “the” is not to be construed as limiting the element to the singular.

Various illustrative logical blocks, modules, components, circuits, and algorithm operations described in connection with the aspects described herein may be implemented as electronic hardware, computer software, or combinations of both. To clearly illustrate this interchangeability of hardware and software, various illustrative components, blocks, modules, circuits, operations, etc. have been described herein generally in terms of their functionality. Whether such functionality is implemented as hardware or software depends upon the particular application and design constraints imposed on the overall system. One of skill in the art may implement the described functionality in varying ways for each particular application, but such implementation decisions should not be interpreted as causing a departure from the scope of the claims.

The hardware used to implement various illustrative logics, logical blocks, modules, components, circuits, etc. described in connection with the aspects described herein may be implemented or performed with a general purpose processor, a digital signal processor (“DSP”), an application specific integrated circuit (“ASIC”), a field programmable gate array (“FPGA”) or other programmable logic device, discrete gate logic, transistor logic, discrete hardware components, or any combination thereof designed to perform the functions described herein. A general-purpose processor may be a microprocessor, a controller, a microcontroller, a state machine, etc. A processor may also be implemented as a combination of receiver smart objects, e.g., a combination of a DSP and a microprocessor, a plurality of microprocessors, one or more microprocessors in conjunction with a DSP core, or any other such like configuration. Alternatively, some operations or methods may be performed by circuitry that is specific to a given function.

In one or more aspects, the functions described may be implemented in hardware, software, firmware, or any combination thereof. If implemented in software, the functions may be stored as one or more instructions (or code) on a non-transitory computer-readable storage medium or a non-transitory processor-readable storage medium. The operations of a method or algorithm disclosed herein may be embodied in a processor-executable software module or as processor-executable instructions, both of which may reside on a non-transitory computer-readable or processor-readable storage medium. Non-transitory computer-readable or processor-readable storage media may be any storage media that may be accessed by a computer or a processor (e.g., RAM, flash, etc.). By way of example but not limitation, such non-transitory computer-readable or processor-readable storage media may include RAM, ROM, EEPROM, NAND FLASH, NOR FLASH, M-RAM, P-RAM, R-RAM, CD-ROM, DVD, magnetic disk storage, magnetic storage smart objects, or any other medium that may be used to store program code in the form of instructions or data structures and that may be accessed by a computer. Disk as used herein may refer to magnetic or non-magnetic storage operable to store instructions or code. Disc refers to any optical disc operable to store instructions or code. Combinations of any of the above are also included within the scope of non-transitory computer-readable and processor-readable media. Additionally, the operations of a method or algorithm may reside as one or any combination or set of codes and/or instructions on a non-transitory processor-readable storage medium and/or computer-readable storage medium, which may be incorporated into a computer program product.

The preceding description of the disclosed aspects is provided to enable any person skilled in the art to make, implement, or use the claims. Various modifications to these aspects will be readily apparent to those skilled in the art, and the generic principles defined herein may be applied to other aspects without departing from the scope of the claims. Thus, the present disclosure is not intended to be limited to the aspects illustrated herein but is to be accorded the widest scope consistent with the claims disclosed herein.

1. An artificial intelligence (“AI”) method for processing and analyzing computer aided dispatch (“CAD”) data to generate stress score data, the method comprising:
receiving, at a processor, CAD incident data from a data agent, the data agent being associated with a CAD system;
storing, at a memory, CAD incident data;
generating, at the processor and using a large-language model (“LLM”) engine, interpreted CAD incident data;
generating, at the processor, stress score data based on the interpreted CAD incident data;
storing, at the memory, stress score data in a database engine; and
presenting, at a user interface, stress score data.

2. The method of claim 1, wherein the storing, at the memory, CAD incident data uses short-term data storage.

3. The method of claim 1, the method further comprising:
associating, at the processor, stress score data with responder profile data; and
presenting, at the user interface, the responder profile data in combination with the stress score data.

4. The method of claim 3, the method further comprising:
generating, at the processor, analytic data associated with the responder profile data and the stress score data; and
presenting, at the user interface, analytic data in combination with the responder profile data.

5. The method of claim 1, wherein the CAD incident data comprises date data, incident type data, disposition data, note data, responder data, or a combination thereof.

6. The method of claim 1, the method further comprising:
defining, at the processor, LLM prompt data; and
configuring, at the processor and using LLM prompt data, the LLM engine to process CAD incident data.

7. The method of claim 6, wherein the LLM prompt data comprises responder mental trauma level data, responder arrival time data, subject status data, subject mental health data, format output type data, or a combination thereof.

8. The method of claim 6, the method further comprising:
evaluating, at the processor, the configuration of the LLM engine in categorizing parsed CAD incident data based on responder mental trauma level data and test CAD incident data; and
presenting, at the user interface, a rationale, at the LLM engine, of the evaluating.

9. The method of claim 7, wherein generating, at the processor, stress score data based on interpreted CAD incident data is further based on responder mental trauma level data within the LLM prompt data.

10. An artificial intelligence (“AI”) system for processing and analyzing computer aided dispatch (“CAD”) data to generate stress score data, the system comprising:
a user interface;
a memory; and
a processor, the processor configured to:
receive CAD incident data from a data agent, the data agent being associated with a CAD system;
store, at the memory, CAD incident data;
generate interpreted CAD incident data;
generate, using a large-language model (“LLM”) engine, stress score data based on the interpreted CAD incident data;
store, at the memory, stress score data in a database engine; and
present, at the user interface, stress score data.

11. The system of claim 10, wherein the storing, at the memory, CAD incident data uses short-term data storage.

12. The system of claim 10, the processor being further configured to:
associate stress score data with responder profile data; and
present, at the user interface, the responder profile data in combination with the stress score data.

13. The system of claim 12, the processor being further configured to:
generate analytic data associated with responder profile data and stress score data; and
present, at the user interface, analytic data in combination with responder profile data.

14. The system of claim 10, wherein the CAD incident data comprises date data, incident type data, disposition data, note data, responder data, or a combination thereof.

15. The system of claim 10, the processor being further configured to:
define LLM prompt data; and
configure, using LLM prompt data, the LLM engine to process CAD incident data.

16. The system of claim 15, wherein the LLM prompt data comprises responder mental trauma level data, responder arrival time data, subject status data, subject mental health data, format output type data, or a combination thereof.

17. The system of claim 15, the processor further configured to:
evaluate the configuration of the LLM engine in categorizing parsed CAD incident data based on responder mental trauma level data and test parsed CAD incident data; and
presenting, at the user interface, a rationale, at the LLM engine, of the evaluating.

18. The system of claim 16, wherein generating stress score data based on the interpreted CAD incident data is further based on the responder mental trauma level data within the LLM prompt data.

19. A computer-readable medium storing instructions that, when executed by a computer, cause the computer to:
define, at a processor, large-language model (“LLM”) prompt data;
configure, at the processor and using LLM prompt data, an LLM engine to process CAD incident data;
receive, at the processor, CAD incident data from a data agent, the data agent being associated with a CAD system;
store, at a memory, CAD incident data;
generate, at the processor, interpreted CAD incident data;
generate, at the processor and using an LLM engine, stress score data based on the interpreted CAD incident data;
store, at the memory, stress score data in a database engine; and
present, at a user interface, stress score data.

20. The computer-readable medium of claim 19, wherein the instructions further cause the computer to:
associate, at the processor, stress score data with responder profile data;
present, at the user interface, the responder profile data in combination with the stress score data;
generate, at the processor, analytic data associated with responder profile data and the stress score data; and
present, at the user interface, analytic data in combination with responder profile data.
